(19) United States
(12) Patent Application Publication
(10) Pub. No.: US 2024/0399289 A1
(43) Pub. Date: Dec. 5, 2024

(54) PYROLYSIS GAS TREATMENT USING ABSORBER-STRIPPER SYSTEM

(71) Applicant: EASTMAN CHEMICAL COMPANY, Kingsport, TN (US)

(72) Inventors: David Eugene Slivensky, Tatum, TX (US); Daryl Bitting, Longview, TX (US); Michael Gary Polasek, Longview, TX (US); Xianchun Wu, Longview, TX (US)

(73) Assignee: Eastman Chemical Company, Kingsport, TN (US)

(21) Appl. No.: 18/691,481

(22) PCT Filed: Sep. 16, 2022
(86) PCT No.: PCT/US2022/043755

§ 371 Date: Mar. 13, 2024

Related U.S. Application Data
(60) Provisional application No. 63/261,424, filed on Sep. 21, 2021.

(51) Int. Cl. B01D 53/14; C07C 7/11; C10B 53/07

(52) U.S. Cl. CPC B01D 53/1425; B01D 53/1431; B01D 53/1475; C07C 7/11; C10B 53/07; B01D 2256/24; B01D 2257/30; B01D 2257/504; B01D 2259/65

(57) ABSTRACT

Processes and facilities for recovering and purifying a pyrolysis gas formed by pyrolyzing waste plastic are provided. An absorber-stripper system may be used to treat the pyrolysis gas for use in one or more downstream chemical recycling processes, which can be used in forming a variety of recycled content products.

### BACKGROUND

Waste plastic pyrolysis plays a part in a variety of chemical recycling technologies. Typically, waste plastic pyrolysis facilities focus on producing recycled content pyrolysis oil (r-pyoil) that can be used in making recycled content products. Waste plastic pyrolysis also produces heavy components (e.g., waxes, tar, and char) and recycled content pyrolysis gas (r-pygas). Although r-pygas produced by the waste plastic pyrolysis typically has 100 percent recycled content, it is common practice for the r-pygas to be burned as fuel to provide heat for the pyrolysis reaction. While burning r-pygas as fuel may be economically efficient, such practice runs counter to one of the main goals of chemical recycling, which is to transform as much of the waste plastic as possible into new products. However, the raw r-pygas stream generally comprises some quantity of carbon dioxide and/or other components that are undesirable for downstream separations and/or other chemical recycling processes.

### SUMMARY

In one aspect, the present technology concerns a process for purifying pyrolysis gas (pygas), the process comprising: (a) pyrolyzing waste plastic to thereby produce a pyrolysis effluent stream; (b) separating at least a portion of the pyrolysis effluent stream to thereby produce a pygas stream and a pyrolysis oil (pyoil) stream; and (c) treating at least a portion of the pygas stream in an absorber-stripper system to thereby produce a purified pygas stream.

In one aspect, the present technology concerns a chemical recycling process, the process comprising: (a) pyrolyzing waste plastic to thereby produce a recycled content pyrolysis gas (r-pygas) stream, wherein the r-pygas stream comprises a quantity of carbon dioxide (CO₂); (b) treating at least a portion of the r-pygas stream in an absorber-stripper system to remove at least a portion of the quantity of CO₂ from the r-pygas stream, thereby producing a CO₂-depleted r-pygas stream; and (c) introducing at least a portion of the CO₂-depleted r-pygas stream into a separation process to thereby produce one or more recycled content chemical product(s) and/or co-product(s).

In one aspect, the present technology concerns a process for recovering heat from a pyrolysis effluent stream, the process comprising: (a) pyrolyzing waste plastic to thereby produce the pyrolysis effluent stream; (b) cooling and at least partially condensing at least a portion of the pyrolysis effluent stream via indirect heat exchange with: (i) a heat transfer medium (HTM), thereby warming the HTM, and/or (ii) a stripper column reboiler; (c) feeding at least a portion of the cooled and at least partially condensed pyrolysis effluent stream to a separator to thereby produce a pyrolysis gas (pygas) stream and a pyrolysis oil (pyoil) stream; (d) treating at least a portion of the pygas stream in an absorber-stripper system to thereby produce a purified pygas stream; and (e) optionally, using at least a portion of the warmed HTM to provide heating to one or more of: (i) a rich solvent stream within the absorber-stripper system; (ii) a liquefication process; and/or (iii) a pyrolysis feedstock preheating process.

### BRIEF DESCRIPTION OF THE DRAWINGS

FIG. 1 is a block flow diagram illustrating the main steps of a process and facility for treating recycled content pyrolysis gas for use in downstream processing to produce recycled chemical products and coproducts; and

FIG. 2 is a block flow diagram illustrating the main steps of a process for recovering a recycled content pyrolysis gas from a pyrolysis effluent and treating the pyrolysis gas in an absorber-stripper system.

### DETAILED DESCRIPTION

We have discovered new methods and systems for utilizing a recycled content stream that was previously burned as fuel. More specifically, we have discovered that pyrolysis gas produced by pyrolyzing waste plastic can be treated, for example in an absorber-stripper system, to be used to produce recycled content products.

As used herein, the term “recycled content” refers to being or comprising a composition that is directly and/or indirectly derived from recycled material, for example recycled waste plastic. Throughout this description, various recycled content components may be denoted by “r-[component].” However, it should be understood that any component that is directly and/or indirectly derived from recycled material may be considered a recycled content component, regardless whether the denotation is used.

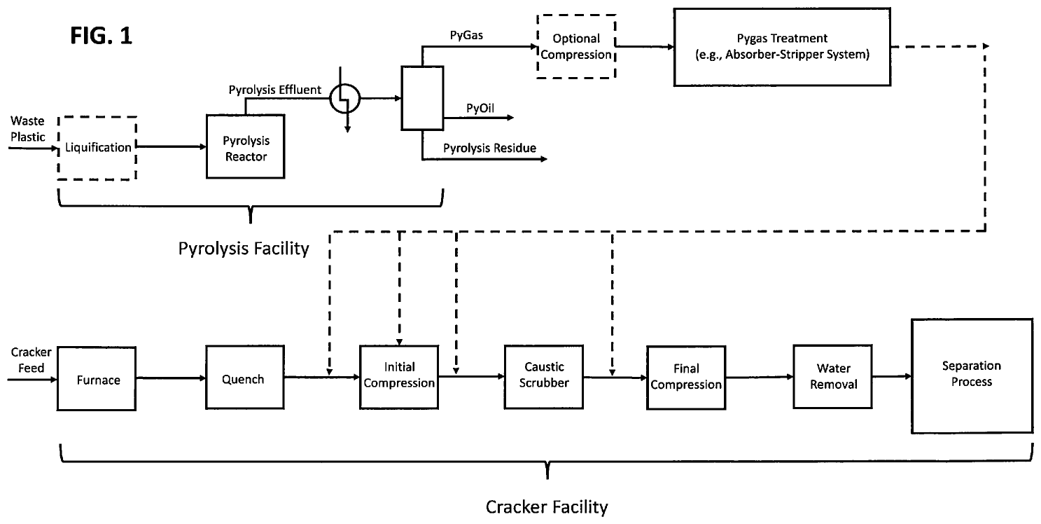

FIG. 1 illustrates one embodiment of a process and system for use in chemical recycling of waste plastic. The process shown in FIG. 1 includes a pyrolysis facility and a cracking facility. The pyrolysis facility and cracking facility may be co-located or located remotely from one another. As used herein, the term “co-located” refers to the characteristic of at least two objects being situated on a common physical site, and/or within 0.5 or 1 mile of each other. As used herein, the term “located remotely” refers to a distance of greater than 1, greater than 5, greater than 10, greater than 50, greater than 100, greater than 500, greater than 1000, or greater than 10,000 miles between two facilities, sites, or reactors.

When two or more facilities are co-located, the facilities may be integrated in one or more ways. Examples of integration include, but are not limited to, heat integration, utility integration, waste-water integration, mass flow integration via conduits, office space, cafeterias, integration of plant management, IT department, maintenance department, and sharing of common equipment and parts, such as seals, gaskets, and the like.

In some embodiments, the pyrolysis facility/process is a commercial scale facility/process receiving the waste plastic feedstock at an average annual feed rate of at least 100, or at least 500, or at least 1,000, at least 2,000, at least 5,000, at least 10,000, at least 50,000, or at least 100,000 pounds per hour, averaged over one year. Further, the pyrolysis facility can produce the r-pyoil and r-pygas in combination at an average annual rate of at least 100, or at least 1,000, or at least 5,000, at least 10,000, at least 50,000, or at least 75,000 pounds per hour, averaged over one year.

Similarly, the cracking facility/process can be a commercial scale facility/process receiving hydrocarbon feed at an average annual feed rate of at least at least 100, or at least 500, or at least 1,000, at least 2,000, at least 5,000, at least 10,000, at least 50,000, or at least 75,000 pounds per hour, averaged over one year. Further, the cracking facility can produce at least one recycled content product stream (r-product) at an average annual rate of at least 100, or at least 1,000, or at least 5,000, at least 10,000, at least 50,000, or at least 75,000 pounds per hour, averaged over one year. When more than one r-product stream is produced, these rates can apply to the combined rate of all r-products and r-coproducts.

As shown in FIG. 1, the process begins by feeding the waste plastic to a pyrolysis facility. In some embodiments, the waste plastic comprises at least 80, at least 90, at least 95, at least 99, or at least 99.9 percent by weight polyolefins. In some embodiments, the waste plastic comprises not more than 10, not more than 5, not more than 1, not more than 0.5, not more than 0.3, not more than 0.2, or not more than 0.1 percent by weight polyesters (e.g., PET). Such low levels of polyesters, such as PET, can be desirable so as to avoid formation of formic acid, acetic acid, others that can cause corrosive compounds to build up in downstream processes.

In some embodiments, the pyrolysis facility comprises a liquification zone for liquifying at least a portion of the waste plastic feed. The liquification zone may comprise a process for liquifying the waste plastic by one or more of: (i) heating/melting; (ii) dissolving in a solvent; (iii) depolymerizing; (iv) plasticizing, and combinations thereof. Additionally, one or more of options (i) through (iv) may also be accompanied by the addition of a blending agent to help facilitate the liquification (reduction of viscosity) of the polymer material.

In some embodiments, the liquification zone includes at least a melt tank and a heater. The melt tank receives the waste plastic feed and the heater heats waste plastic stream. The melt tank can include one or more continuously stirred tanks. When one or more rheology modification agents (e.g., solvents, depolymerization agents, plasticizers, and blending agents) are used in the liquification zone, such rheology modification agents can be added to and/or mixed with the waste plastic in the melt tank. The heater of the liquification zone can take the form of internal heat exchange coils located in the melt tank and/or an external heat exchanger. The heater may transfer heat to the waste plastic via indirect heat exchange with a process stream or heat transfer medium, such as in the heat integration processes described in greater detail below.

Within the pyrolysis facility, the waste plastic or liquified waste plastic is fed to a pyrolysis step where the waste plastic is pyrolyzed in a pyrolysis reactor. The pyrolysis reaction involves chemical and thermal decomposition of the waste plastic introduced into the reactor. Although all pyrolysis processes may be generally characterized by a reaction environment that is substantially free of oxygen, pyrolysis processes may be further defined, for example, by the pyrolysis reaction temperature within the reactor, the residence time in the pyrolysis reactor, the reactor type, the pressure within the pyrolysis reactor, and the presence or absence of pyrolysis catalysts. The pyrolysis reactor can be, for example, a film reactor, a screw extruder, a tubular reactor, a tank, a stirred tank reactor, a riser reactor, a fixed bed reactor, a fluidized bed reactor, a rotary kiln, a vacuum reactor, a microwave reactor, or an autoclave.

The pyrolysis reaction can involve heating and converting the waste plastic feedstock in an atmosphere that is substantially free of oxygen or in an atmosphere that contains less oxygen relative to ambient air. For example, the atmosphere within the pyrolysis reactor may comprise not more than 5, not more than 4, not more than 3, not more than 2, not more than 1, or not more than 0.5 weight percent of oxygen.

In one embodiment or in combination with one or more embodiments disclosed herein, the pyrolysis reaction performed in the pyrolysis reactor can be carried out at a temperature of less than 700° C., less than 650° C., or less than 600° C. and at least 300° C., at least 350° C., or at least 400° C. The feed to the pyrolysis reactor can comprise, consists essentially of, or consists of waste plastic. The feed stream, and/or the waste plastic component of the feed stream, can have a number average molecular weight (Mn) of at least 3000, at least 4000, at least 5000, or at least 6000 g/mole. If the feed to the pyrolysis reactor contains a mixture of components, the Mn of the pyrolysis feed is the weighted average Mn of all feed components, based on the mass of the individual feed components. The waste plastic in the feed to the pyrolysis reactor can include post-consumer waste plastic, post-industrial waste plastic, or combinations thereof. In certain embodiments, the feed to the pyrolysis reactor comprises less than 5, less than 2, less than 1, less than 0.5, or about 0.0 weight percent coal and/or biomass (e.g., lignocellulosic waste, switchgrass, fats and oils derived from animals, fats and oils derived from plants, etc.), based on the weight of solids in pyrolysis feed or based on the weight of the entire pyrolysis feed. The feed to the pyrolysis reaction can also comprise less than 5, less than 2, less than 1, or less than 0.5, or about 0.0 weight percent of a co-feed stream, including steam, sulfur-containing co-feed streams, and/or non-plastic hydrocarbons (e.g., non-plastic hydrocarbons having less than 50, less than 30, or less than 20 carbon atoms), based on the weight of the entire pyrolysis feed other than water or based on the weight of the entire pyrolysis feed. The reactor may also utilize a feed gas and/or lift gas for facilitating the introduction of the feed into the pyrolysis reactor. The feed gas and/or lift gas can comprise nitrogen and can comprise less than 5, less than 2, less than 1, or less than 0.5, or about 0.0 weight percent of steam and/or sulfur-containing compounds.

The temperature in the pyrolysis reactor can be adjusted to facilitate the production of certain end products. In some embodiments, the peak pyrolysis temperature in the pyrolysis reactor can be at least 325° C., or at least 350° C., or at least 375° C., or at least 400° C. Additionally or alternatively, the peak pyrolysis temperature in the pyrolysis reactor can be not more than 800° C., not more than 700° C., or not more than 650° C., or not more than 600° C., or not more than 550° C., or not more than 525° C., or not more than 500° C., or not more than 475° C., or not more than 450° C., or not more than 425° C., or not more than 400° C. More particularly, the peak pyrolysis temperature in the pyrolysis reactor can range from 325 to 800° C., or 350 to 600° C., or 375 to 500° C., or 390 to 450° C., or 400 to 500° C.

The residence time of the feedstock within the pyrolysis reactor can be at least 1, or at least 5, or at least 10, or at least 20, or at least 30, or at least 60, or at least 180 seconds. Additionally, or alternatively, the residence time of the feedstock within the pyrolysis reactor can be less than 2, or less than 1, or less than 0.5, or less than 0.25, or less than 0.1 hours. More particularly, the residence time of the feedstock within the pyrolysis reactor can range from 1 second to 1 hour, or 10 seconds to 30 minutes, or 30 seconds to 10 minutes.

The pyrolysis reactor can be maintained at a pressure of at least 0.1, or at least 0.2, or at least 0.3 barg and/or not more than 60, or not more than 50, or not more than 40, or not more than 30, or not more than 20, or not more than 10, or not more than 8, or not more than 5, or not more than 2, or not more than 1.5, or not more than 1.1 barg. The pressure within the pyrolysis reactor can be maintained at atmospheric pressure or within the range of 0.1 to 60, or 0.2 to 10, or 0.3 to 1.5 barg.

The pyrolysis reaction in the reactor can be thermal pyrolysis, which is carried out in the absence of a catalyst, or catalytic pyrolysis, which is carried out in the presence of a catalyst. When a catalyst is used, the catalyst can be homogenous or heterogeneous and may include, for example, certain types of zeolites and other mesostructured catalysts.

As shown in FIG. 1, a pyrolysis effluent is produced and removed from the reactor and generally comprises pyrolysis oil (pyoil), pyrolysis gas (pygas), and pyrolysis residue. As used herein, the terms “pyrolysis gas” or “pygas” refers to a composition obtained from waste plastic pyrolysis that is gaseous at 25° C. at 1 atm. As used herein, the terms “pyrolysis oil” or “pyoil” refers to a composition obtained from waste plastic pyrolysis that is liquid at 25° C. and 1 atm. As used herein, the term “pyrolysis residue” refers to a composition obtained from waste plastic pyrolysis that is not pygas or pyoil and that comprises predominantly pyrolysis char and pyrolysis heavy waxes. As used herein, the term “pyrolysis char” refers to a carbon-containing composition obtained from pyrolysis that is solid at 200° C. and 1 atm. As used herein, the term “pyrolysis heavy waxes” refers to C20+ hydrocarbons obtained from pyrolysis that are not pyrolysis char, pyrolysis gas, or pyrolysis oil.

In some embodiments, the pyrolysis effluent may comprise in the range of 20 to 99 weight percent, 25 to 80 weight percent, 30 to 85, 30 to 80, 30 to 75, 30 to 70, or 30 to 65 weight percent of the pyrolysis oil. In some embodiments, the pyrolysis effluent may comprise 1 to 90, 10 to 85, 15 to 85, 20 to 80, 25 to 80, 30 to 75, or 35 to 75 weight percent of the pyrolysis gas. In some embodiments, the pyrolysis effluent may comprise in the range of 0.1 to 25, 1 to 15, 1 to 8, or 1 to 5 weight percent of the pyrolysis residue.

In some embodiments, the pyrolysis effluent may comprise not more than 15, not more than 10, not more than 5, not more than 2, not more than 1, or not more than 0.5 weight percent of free water. As used herein, the term “free water” refers to water previously added to the pyrolysis unit and water generated in the pyrolysis unit.

The pyrolysis effluent generally leaves the pyrolysis reactor at very high temperatures (e.g., 500° C. to 800° C.) and thus must be cooled and at least partially condensed before being separated into respective pyrolysis gas, pyrolysis oil, and pyrolysis residue streams. The heat from the pyrolysis effluent can therefore be recovered and used in various processes throughout the chemical recycling process.

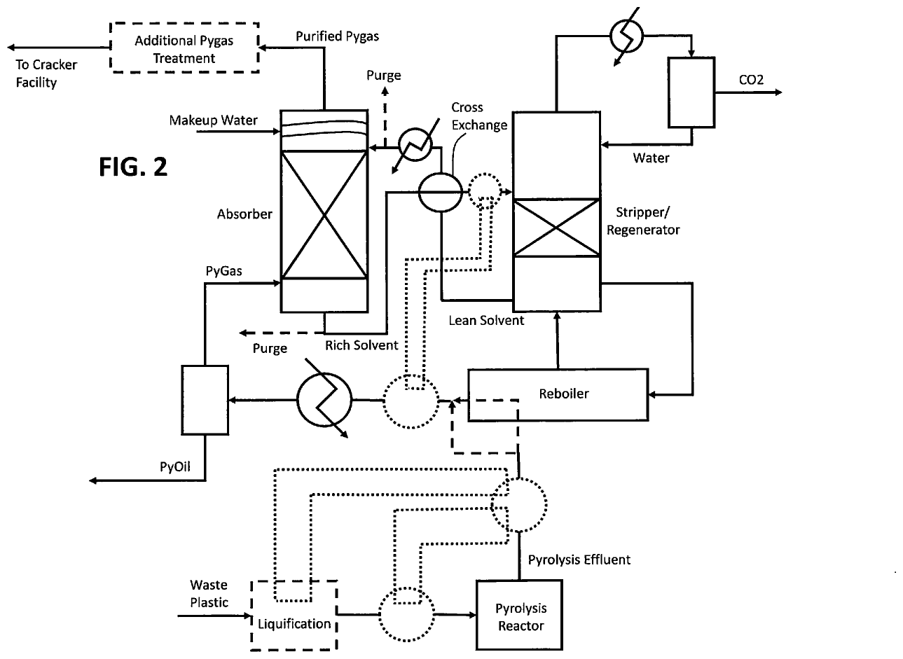

Referring now to FIG. 2, various potential pyrolysis effluent heat recovery processes are shown. In some embodiments, pyrolysis effluent stream can be cooled via indirect heat exchange with one or more heat transfer medium (HTM) streams, thereby warming the HTM, and/or by providing heat to a stripper column reboiler, for example in an absorber-stripper system, as described below. In some embodiments, the HTM comprises water/steam, oils, siloxanes, molten metal, molten salt, and/or combinations thereof. The HTM may comprise an oil selected from the group consisting of synthetic oil, refined oil (e.g., mineral oil), or a combination thereof. As used herein, the term “refined oil” refers to a natural (i.e., non-synthetic) oil that has been subjected to a distillation and/or or purification step.

As shown in FIG. 2, the warmed HTM stream(s) may be used to provide heating to optionally, using at least a portion of the warmed HTM to provide heating to one or more of a rich solvent stream within an absorber-stripper system, a liquefication process (e.g., melt tank heating and/or preheating upstream of the melt tank), and/or a pyrolysis feedstock preheating process. In some embodiments, further cooling may be necessary to cool the pyrolysis effluent stream to an appropriate temperature to separate the pyrolysis gas, pyrolysis oil, and pyrolysis residue. For example, in some embodiments, the pyrolysis effluent stream is cooled to a temperature of not more than 60° C., or not more than 50° C. before being fed to the separator. In some embodiments, the pyrolysis effluent stream is cooled to a temperature of 15° C. to 60° C., 25° C. to 45° C., or 30° C. to 40° C. before being fed to the separator.

After cooling, the pyrolysis effluent stream may be fed to a separator to thereby produce a pyrolysis gas (pygas) stream, a pyrolysis (pyoil) stream, and a pyrolysis residue stream. In some embodiments, the pygas stream comprises 1 to 50 weight percent methane and/or 5 to 99 weight percent C2, C3, and/or C4 hydrocarbon content (including all hydrocarbons having 2, 3, or 4 carbon atoms per molecule). The pygas stream may comprise C2 and/or C3 components each in an amount of 5 to 60, 10 to 50, or 15 to 45 weight percent, C4 components in an amount of 1 to 60, 5 to 50, or 10 to 45 weight percent, and C5 components in an amount of 1 to 25, 3 to 20, or 5 to 15 weight percent. The pyrolysis gas may have a temperature of 15° C. to 60° C., 25° C. to 45° C., or 30° C. to 40° C. before treatment (described below).

In some embodiments, the pyoil stream comprises at least 50, at least 75, at least 90, or at least 95 weight percent of C4 to C30, C5 to C25, C5 to C22, or C5 to C20 hydrocarbon components. The pyoil can have a 90% boiling point in the range of from 150 to 350° C., 200 to 295° C., 225 to 290° C., or 230 to 275° C. As used herein, “boiling point” refers to the boiling point of a composition as determined by ASTM D2887-13. Additionally, as used herein, an “90% boiling point,” refers to a boiling point at which 90 percent by weight of the composition boils per ASTM D-2887-13.

In some embodiments, the pyoil can comprise heteroatom-containing compounds in an amount of less than 20, less than 10, less than 5, less than 2, less than 1, or less than 0.5 weight percent. As used herein, the term “heteroatom-containing” compound includes any compound or polymer containing nitrogen, sulfur, or phosphorus. Any other atom is not regarded as a “heteroatom” for purposes of determining the quantity of heteroatoms, heterocompounds, or heteropolymers present in the pyoil. Heteroatom-containing compounds include oxygenated compounds. Often, such compounds exist in r-pyoil when the pyrolyzed waste plastic includes polyethylene terephthalate (PET) and/or polyvinyl chloride (PVC). Thus, little to no PET and/or PVC in the waste plastic 110 results in little to no heteroatom-containing compounds in the pyoil.

As shown in FIG. 1, the pygas stream from the pyrolysis effluent separator may be fed to an optional compression zone prior to being introduced to one or more pygas treatment processes. This optional compression zone may include one or more compressors, followed by one or more coolers, and/or a liquid knockout section. In some embodiments, the one or more pygas treatment processes comprise a carbon dioxide removal process, a halogen removal process, and/or a sulfur removal process.

Referring again to FIG. 2, in some embodiments, the one or more pygas treatment processes may comprise an absorber-stripper system. Although only a single absorber tower and regeneration tower are shown in FIG. 2, the absorber-stripper system can comprise one or more absorber towers and one or more regeneration towers. The absorber tower(s) and regeneration tower(s) may be constructed to appropriate size and specification as understood in the art, based on the pygas composition and flow rate and the absorber solvent used.

To treat the pygas, the pygas stream is introduced into the one or more absorber towers, where the pygas contacts an absorber solvent (i.e., a lean absorber solvent) that is concurrently introduced into the one or more absorber towers. Upon contact, at least a portion of the carbon dioxide and/or other impurities in the pygas stream is absorbed and removed in the rich absorber solvent stream. In some embodiments, the absorber solvent comprises a component selected from the group consisting of amines, methanol, sodium hydroxide, sodium carbonate/bicarbonate, potassium hydroxide, potassium carbonate/bicarbonate, SELEXOL®, glycol ether, and combinations thereof. In some embodiments, the absorber solvent can comprise an absorbing component selected from the group consisting of amines, methanol, SELEXOL®, glycol ether, and combinations thereof. The absorbing component may comprise an amine selected from the group consisting of diethanolamine (DEA), monoethanolamine (MEA), methyldiethanolamine (MDEA), diisopropanolamine (DIPA), diglycolamine (DGA), piperazine, modifications, derivatives, and combinations thereof.

The resulting purified pygas exits the absorber tower(s) overhead and is generally depleted in carbon dioxide relative to the pygas stream fed into the absorber tower(s). In some embodiments, the purified pygas stream comprises not more than 1000 ppm, not more than 500 ppm, not more than 400 ppm, not more than 300 ppm, not more than 200 ppm, or not more than 100 ppm carbon dioxide. In some embodiments, the purified pygas stream is also depleted in sulfur and/or sulfur-containing compounds (e.g., H₂S) relative to the pygas stream fed into the absorber tower(s).

In some embodiments, the purified pygas stream has a temperature of not more than 60° C. after treating in the absorber system. The purified pygas stream may have a temperature of 45° C. to 60° C., or 50° C. to 55° C. after treating in the absorber system. The purified pygas stream may have a temperature of 1° to 40°, 5° to 30°, or 10° to 20° greater than the pygas stream before being fed into the absorber tower(s).

The absorbed carbon dioxide can be removed from the absorber solvent in the regeneration tower(s). Within the regeneration tower(s), the carbon dioxide can be stripped from the rich solvent by contacting the solvent with water/steam. The overhead stream comprising steam and carbon dioxide is then cooled and at least partially condensed to remove the carbon dioxide gas, and the water component is recycled back into the regeneration tower(s).

The one or more regeneration towers generally comprise at least one reboiler, which operates at a temperature high enough to release the carbon dioxide from the absorber solvent but below the degradation temperature of the absorber solvent. In some embodiments, the reboiler operates at a temperature of 105° C. to 130° C., 110° C. to 125° C., or 115° C. to 120° C.

The absorber-stripper system may further comprise one or more additional components or processes as understood in the art for appropriate operation of the system. For example, in some embodiments, a cross-heat exchanger may be utilized to provide appropriate heating and cooling to the absorber solvent. In some embodiments, one or more purge outlets may be included to remove excess solvent, water, or other components from the system. However, such components may also be purged using a reclaimer or temporarily shutting down the system.

As shown in FIG. 1, at least a portion of the purified pygas can be introduced into a cracker facility. In some embodiments, at least 50, at least 75, at least 90, or at least 95 percent of the pygas from the pyrolysis facility can be introduced into the cracker facility after treatment in the form of the purified pygas. Additionally, or alternatively, all or a portion of the purified pygas may be introduced into at least one location downstream of the cracker furnace.

When introduced into a location downstream of the cracker furnace, the purified pygas may be introduced into one or more of the following locations: (i) upstream of the initial compression zone, which compresses the vapor portion of the furnace effluent in two or more compression stages; (ii) into the initial compression zone, between individual compressors; (iii) downstream of the initial compression zone but upstream of a caustic scrubber process; and/or (iv) downstream of the caustic scrubber process but upstream of the final compression zone. In some cases, the purified pygas may be introduced into only one of these locations, while, in other cases, the purified pygas may be divided into additional fractions and each fraction introduced into a different location. In such cases, the fractions of the purified pygas may be introduced into at least two, three, or all of the locations shown in FIG. 1.

The location where the purified pygas stream may be introduced into the cracker facility may depend on the pressure of the pygas stream, which will depend on whether a compression zone is used upstream of the pygas treatment and the conditions of the pygas treatment process(es). For example, if there is no compression zone upstream of the pygas treatment, then the purified pygas stream may need to be introduced upstream of the initial compression section of the cracker facility. However, if there is a compression zone upstream of the pygas treatment, then the purified pygas stream may be introduced into a location downstream of the initial compression section of the cracker facility.

When introduced into the initial compression section, the purified pygas may be introduced upstream of the first compression stage, upstream or downstream of the last compression stage, or upstream of one or more intermediate compression stages.

When introduced upstream of the caustic scrubber process, the purified pygas can be fed along with the cracker effluent into the caustic scrubber to further remove carbon dioxide, sulfur, and/or other impurities from the pygas stream.

The cracker facility process generally comprises feeding a hydrocarbon feed into the inlet of a cracker furnace. The hydrocarbon feed may comprise predominantly C3 to C5 hydrocarbon components, C5 to C22 hydrocarbon components, or C3 to C22 hydrocarbon components, or even predominantly C2 components. The hydrocarbon feed may include recycled content from one or more sources, or it may include non-recycled content. Additionally, in some cases, the hydrocarbon feed may not include any recycled content.

In one embodiment or in combination with one or more embodiments disclosed herein, the cracker furnace can be operated at a product outlet temperature (e.g., coil outlet temperature) of at least 700° C., at least 750° C., at least 800° C., or at least 850° C. The feed to the cracker furnace can have a number average molecular weight (Mn) of less than 3000, less than 2000, less than 1000, or less than 500 g/mole. If the feed to the cracker furnace contains a mixture of components, the Mn of the cracker feed is the weighted average Mn of all feed components, based on the mass of the individual feed components. The feed to the cracker furnace can comprise less than 5, less than 2, less than 1, less than 0.5, or 0.0 weight percent of coal, biomass, and/or solids. In certain embodiments, a co-feed stream, such as steam or a sulfur-containing stream (for metal passivation) can be introduced into the cracker furnace. The cracker furnace can include both convection and radiant sections and can have a tubular reaction zone (e.g., coils in one or both of the convection and radiant sections). Typically, the residence time of the streams passing through the reaction zone (from the convection section inlet to the radiant section outlet) can be less than 20 seconds, less than 10 seconds, less than 5 seconds, or less than 2 seconds.

The hydrocarbon feed can be thermally cracked within the furnace to form a lighter hydrocarbon effluent. The effluent stream can then be cooled in the quench zone and compressed in the compression zone. The compressed stream from the compression zone can then be fed as a cracked gas stream to a caustic scrubber process and then be further separated in the separation zone to produce at least one recycled content chemical product (r-product) and/or coproduct(s). Examples of recycled content products and coproducts include, but are not limited to, recycled content ethane (r-ethane), recycled content ethylene (r-ethylene), recycled content propane (r-propane), recycled content propylene (r-propylene), recycled content butane (r-butane), recycled content butenes (r-butenes), recycled content butadiene (r-butadiene), and recycled content pentanes and heavier (r-C5+). In some embodiments, at least a portion of the recycled content stream (e.g., r-ethane or r-propane) may be returned to the inlet of the cracker furnace as a reaction recycle stream.

When the one or more purified pygas streams are introduced into the cracking facility, the purified pygas may be combined with at least a portion of the cracker effluent (e.g., as compressed cracked gas), and the combined gas stream may be fed to a caustic scrubber process and/or otherwise processed in the same or similar manner as the cracked gas described above. For example, in some embodiments, the gas stream can be introduced into a separation zone (either directly or indirectly via one or more locations within the cracker facility). Thus, the purified pygas can be used to produce various recycled content chemical products and coproducts, which may be the same or different from those described above. In some embodiments, the recycled content chemical product(s) and co-product(s) comprise olefins (e.g., C2-C5 alkenes), alkanes (e.g., C2-C5 alkanes), aromatics (e.g., benzene, toluene, xylenes, styrene), hydrogen (H2), paraffins, gasoline, and/or C5+ hydrocarbons. In some embodiments, the recycled content product(s) and co-product(s) comprise r-ethylene, r-propylene, r-butylene, r-benzene, r-toluene, r-xylenes, and/or r-styrene.

### Definitions

It should be understood that the following is not intended to be an exclusive list of defined terms. Other definitions may be provided in the foregoing description, such as, for example, when accompanying the use of a defined term in context.

Unless otherwise expressly stated, all “ppm” and “ppb” values expressed are by weight with respect to liquids and solids, and by volume with respect to gases. For multi-phase streams, “ppm” and “ppb” values expressed for components primarily in the gaseous phase are by volume, and “ppm” and “ppb” values expressed for components primarily in the liquid and/or solids phases are by weight.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and propylene) that are useful by themselves and/or are useful as feedstocks to another chemical production process(es).

As used herein, the term “co-located” refers to the characteristic of at least two objects being situated on a common physical site, and/or within one mile of each other.

As used herein, the term “commercial scale facility” refers to a facility having an average annual feed rate of at least 500 pounds per hour, averaged over one year.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the term “cracking” refers to breaking down complex organic molecules into simpler molecules by the breaking of carbon-carbon bonds.

As used herein, the term “depleted” refers to having a concentration of a specific component that is less than the concentration of that component in a reference material or stream.

As used herein, the term “enriched” refers to having a concentration of a specific component that is greater than the concentration of that component in a reference material or stream.

As used herein, the term “free water” refers to water previously added (as liquid or steam) to the pyrolysis unit and water generated in the pyrolysis unit.

As used herein, the term “halogen” or “halogens” refers to organic or inorganic compounds, ionic, or elemental species comprising at least one halogen atom.

As used herein, the terms “including,” “include,” and “included” have the same open-ended meaning as “comprising,” “comprises,” and “comprise” provided above.

As used herein, the term “located remotely” refers to a distance of greater than 1, 5, 10, 50, 100, 500, 1000, or 10,000 miles between two facilities, sites, or reactors. As used herein, the term “predominantly” means more than 50 percent by weight. For example, a predominantly propane stream, composition, feedstock, or product is a stream, composition, feedstock, or product that contains more than 50 weight percent propane.

As used herein, the term “pyrolysis” refers to thermal decomposition of one or more organic materials at elevated temperatures in an inert (i.e., substantially oxygen free) atmosphere.

As used herein, the terms “pyrolysis gas” and “pygas” refer to a composition obtained from pyrolysis that is gaseous at 25° C.

As used herein, the terms “pyrolysis oil” or “pyoil” refers to a composition obtained from pyrolysis that is liquid at 25° C. and 1 atm.

As used herein, the term “pyrolysis residue” refers to a composition obtained from pyrolysis that is not pyrolysis gas or pyrolysis oil and that comprises predominantly pyrolysis char and pyrolysis heavy waxes.

As used herein, the term “recycled content” refers to being or comprising a composition that is directly and/or indirectly derived from recycled material.

As used herein, the term “refined oil” refers to a natural (i.e., non-synthetic) oil that has been subjected to a distillation and/or or purification step.

As used herein, the term “waste material” refers to used, scrap, and/or discarded material.

As used herein, the terms “waste plastic” and “plastic waste” refer to used, scrap, and/or discarded plastic materials.

### Additional Claim Supporting Description—First Embodiment

In a first embodiment of the present technology there is provided a process for purifying pyrolysis gas (pygas), the process comprising: (a) pyrolyzing waste plastic to thereby produce a pyrolysis effluent stream; (b) separating at least a portion of the pyrolysis effluent stream to thereby produce a pygas stream and a pyrolysis oil (pyoil) stream; and (c) treating at least a portion of the pygas stream in an absorber-stripper system to thereby produce a purified pygas stream.

The first embodiment described in the preceding paragraph can also include one or more of the additional aspects/features listed in the following bullet pointed paragraphs. Each of the below additional features of the first embodiment can be standalone features or can be combined with one or more of the other additional features to the extent consistent. Additionally, the following bullet pointed paragraphs can be viewed as dependent claim features having levels of dependency indicated by the degree of indention in the bulleted list (i.e., a feature indented further than the feature(s) listed above it is considered dependent on the feature(s) listed above it).
Wherein the absorber-stripper system comprises one or more absorber towers and one or more regeneration towers.
   Wherein the treating (c) comprises introducing the pygas stream to the one or more absorber towers and contacting the pygas with an absorber solvent.
      Wherein the absorber solvent comprises an absorbing component selected from the group consisting of amines, methanol, sodium hydroxide, sodium carbonate/bicarbonate, potassium hydroxide, potassium carbonate/bicarbonate, SELEXOL®, glycol ether, and combinations thereof.
         Wherein the absorbing component comprises an amine selected from the group consisting of diethanolamine (DEA), monoethanolamine (MEA), methyldiethanolamine (MDEA), diisopropanolamine (DIPA), diglycolamine (DGA), piperazine, modifications, derivatives, and combinations thereof.
         Wherein the one or more regeneration towers comprise at least one reboiler operating at a temperature high enough to release the CO₂ from the absorber solvent and below the degradation temperature of the absorber solvent.
         Wherein the reboiler operates at a temperature of 105° C. to 130° C., 110° C. to 125° C., or 115° C. to 120° C.
Further comprising cooling the pyrolysis effluent stream to a temperature not more than 50° C. before treating in the absorber-stripper system.
   Further comprising cooling the pyrolysis effluent stream to a temperature of 15° C. to 60° C., 25° C. to 45° C., or 30° C. to 40° C. before treating in the absorber-stripper system.
Wherein the purified pygas stream comprises not more than 1000 ppm, 500 ppm, 400 ppm, 300 ppm, 200 ppm, or 100 ppm CO₂.
Wherein the purified pygas stream has a temperature of not more than 60° C. after treating in the absorber system.
Wherein the purified pygas stream has a temperature of 45° C. to 60° C., or 50° C. to 55° C. after treating in the absorber system.
Wherein the purified pygas stream has a temperature of 1° to 40°, 5° to 30°, or 10° to 20° greater than the pygas stream before the treating (c).
Wherein the purified pygas stream is depleted in carbon dioxide (CO₂) relative to the pygas stream fed to the absorber-stripper system.
Wherein the purified pygas stream is depleted in sulfur and/or sulfur-containing compounds (e.g., H₂S).
Wherein the waste plastic comprises not more than 10, not more than 5, not more than 1, not more than 0.5, not more than 0.3, not more than 0.2, or not more than 0.1 percent by weight polyesters (e.g., PET).
Wherein the waste plastic comprises at least 80, at least 90, at least 95, at least 99, or at least 99.9 percent by weight polyolefins.
Wherein the pyrolysis effluent comprises:
   20 to 99 weight percent pyoil;
   1 to 90 weight percent pygas;
   0.1 to 25 weight percent pyrolysis residue; and/or
   not more than 15, 10, 5, 2, 1, 0.5 weight percent of free water.
Wherein the pygas stream (i.e., before treatment) comprises:
   1 to 50 weight percent methane.
5 to 99 weight percent C2, C3, and/or C4 hydrocarbon content

### Additional Claim Supporting Description—Second Embodiment

In a second embodiment of the present technology there is provided a chemical recycling process, the process comprising: (a) pyrolyzing waste plastic to thereby produce a recycled content pyrolysis gas (r-pygas) stream, wherein the r-pygas stream comprises a quantity of carbon dioxide (CO₂); (b) treating at least a portion of the r-pygas stream in an absorber-stripper system to remove at least a portion of the quantity of CO₂ from the r-pygas stream, thereby producing a CO₂-depleted r-pygas stream; and (c) introducing at least a portion of the CO₂-depleted r-pygas stream into a separation process to thereby produce one or more recycled content chemical product(s) and/or co-product(s).

The second embodiment described in the preceding paragraph can also include one or more of the additional aspects/features listed in the following bullet pointed paragraphs. Each of the below additional features of the second embodiment can be standalone features or can be combined with one or more of the other additional features to the extent consistent. Additionally, the following bullet pointed paragraphs can be viewed as dependent claim features having levels of dependency indicated by the degree of indention in the bulleted list (i.e., a feature indented further than the feature(s) listed above it is considered dependent on the feature(s) listed above it).
Wherein the recycled content chemical product(s) and co-product(s) comprise olefins (e.g., C2-C5 alkenes), alkanes (e.g., C2-C5 alkanes), aromatics (e.g., benzene, toluene, xylenes, styrene), hydrogen (H2), paraffins, gasoline, and/or C5+ hydrocarbons.
Wherein the r-Chemical product(s) and co-product(s) comprises r-ethylene, r-propylene, r-butylene, r-benzene, r-toluene, r-xylenes, and/or r-styrene.
Wherein the absorber-stripper system comprises a regenerable absorber process comprising one or more absorber tower(s) and one or more regeneration tower(s).
   Wherein the treating (b) comprises introducing the r-pygas stream to the absorber tower(s) and contacting the r-pygas with an absorber solvent.
      Wherein the absorber solvent comprises an absorbing component selected from the group consisting of amines, methanol, sodium hydroxide, sodium carbonate/bicarbonate, potassium hydroxide, potassium carbonate/bicarbonate, SELEXOL®, glycol ether, and combinations thereof.
         Wherein the absorbing component comprises an amine selected from the group consisting of diethanolamine (DEA), monoethanolamine (MEA), methyldiethanolamine (MDEA), diisopropanolamine (DIPA), diglycolamine (DGA), piperazine, modifications, derivatives, and combinations thereof.
         Wherein the regeneration tower(s) comprise a reboiler operating at a temperature high enough to release the CO₂ from the absorber solvent and below the degradation temperature of the absorbing component.
         Wherein the reboiler operates at a temperature of 105° C. to 130° C., 110° C. to 125° C., or 115° C. to 120° C.
Wherein the CO₂-depleted r-pygas stream comprises not more than 1000 ppm, 500 ppm, 400 ppm, 300 ppm, 200 ppm, or 100 ppm CO₂.
Wherein the CO₂-depleted r-pygas stream has a temperature of not more than 60° C. after treating in the absorber system.
Wherein the CO₂-depleted r-pygas stream has a temperature of 45° C. to 60° C., or 50° C. to 55° C. after treating in the absorber system.
Wherein the CO₂-depleted r-pygas stream has a temperature of 1° to 40°, 5° to 30°, or 10° to 20° greater than the PyGas stream before the treating (c).
Wherein the CO₂-depleted r-pygas stream is depleted in sulfur and/or sulfur-containing compounds (e.g., H₂S).
Wherein the waste plastic comprises not more than 10, not more than 5, not more than 1, not more than 0.5, not more than 0.3, not more than 0.2, or not more than 0.1 percent by weight polyesters (e.g., PET).
Wherein the waste plastic comprises at least 80, at least 90, at least 95, at least 99, or at least 99.9 percent by weight polyolefins.

### Additional Claim Supporting Description—Third Embodiment

In a third embodiment of the present technology there is provided a process for recovering heat from a pyrolysis effluent stream, the process comprising: (a) pyrolyzing waste plastic to thereby produce the pyrolysis effluent stream; (b) cooling and at least partially condensing at least a portion of the pyrolysis effluent stream via indirect heat exchange with: (i) a heat transfer medium (HTM), thereby warming the HTM, and/or (ii) a stripper column reboiler; (c) feeding at least a portion of the cooled and at least partially condensed pyrolysis effluent stream to a separator to thereby produce a pyrolysis gas (pygas) stream and a pyrolysis oil (pyoil) stream; (d) treating at least a portion of the pygas stream in an absorber-stripper system to thereby produce a purified pygas stream; and (e) optionally, using at least a portion of the warmed HTM to provide heating to one or more of: (i) a rich solvent stream within the absorber-stripper system; (ii) a liquefication process; and/or (iii) a pyrolysis feedstock preheating process.

The third embodiment described in the preceding paragraph can also include one or more of the additional aspects/features listed in the following bullet pointed paragraphs. Each of the below additional features of the third embodiment can be standalone features or can be combined with one or more of the other additional features to the extent consistent. Additionally, the following bullet pointed paragraphs can be viewed as dependent claim features having levels of dependency indicated by the degree of indention in the bulleted list (i.e., a feature indented further than the feature(s) listed above it is considered dependent on the feature(s) listed above it).
Wherein the pyrolysis effluent stream has a temperature of 500° C. to 800° C. after the pyrolyzing.
Wherein the heat transfer medium (HTM) comprises water/steam, oils, siloxanes, molten metal, molten salt, and/or combinations thereof.
   Wherein the HTM comprises an oil selected from the group consisting of synthetic oil, refined oil (e.g., mineral oil), or a combination thereof.
Wherein the reboiler operates at a temperature of 105° C. to 130° C., 110° C. to 125° C., or 115° C. to 120° C.
Further comprising cooling the pyrolysis effluent stream to a temperature not more than 60° C. before treating in the absorber system.
Further comprising cooling the pyrolysis effluent stream to a temperature of 15° C. to 60° C., 25° C. to 45° C., or 30° C. to 40° C. before treating in the absorber-stripper system.

### Claims not Limited to Disclosed Embodiments

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A process for purifying pyrolysis gas (pygas), the process comprising:
(a) pyrolyzing waste plastic to thereby produce a pyrolysis effluent stream;
(b) separating at least a portion of the pyrolysis effluent stream to produce a pygas stream and a pyrolysis oil (pyoil) stream; and
(c) treating at least a portion of the pygas stream in an absorber-stripper system to produce a purified pygas stream.

2. The process of claim 1, wherein the absorber-stripper system comprises one or more absorber towers and one or more regeneration towers.

3. The process of claim 2, wherein the treating (c) comprises introducing the pygas stream to the one or more absorber towers and contacting the pygas with an absorber solvent.

4. The process of claim 3, wherein the absorber solvent comprises a component selected from the group consisting of amines, methanol, sodium hydroxide, sodium carbonate/bicarbonate, potassium hydroxide, potassium carbonate/bicarbonate, SELEXOL®, glycol ether, and combinations thereof.

5. The process of claim 4, wherein the absorber solvent comprises an amine selected from the group consisting of diethanolamine (DEA), monoethanolamine (MEA), methyldiethanolamine (MDEA), diisopropanolamine (DIPA), diglycolamine (DGA), piperazine, modifications, derivatives, and combinations thereof.

6. The process of claim 3, wherein the one or more regeneration towers comprise at least one reboiler operating at a temperature high enough to release the CO₂ from the absorber solvent and below the degradation temperature of the absorber solvent.

7. The process of claim 6, wherein the stripper column reboiler operates at a temperature of 105° C. to 130° C.

8. The process of claim 1, further comprising cooling the pyrolysis effluent stream to a temperature not more than 60° C. before the separating (b).

9. The process of claim 1, wherein after the treating (c), the purified pygas stream has a temperature of 1° to 40° greater than the temperature of the pygas stream before the treating (c).

10. A chemical recycling process, the process comprising:
(a) pyrolyzing waste plastic to produce a recycled content pyrolysis gas (r-pygas) stream, wherein the r-pygas stream comprises a quantity of carbon dioxide (CO₂);
(b) treating at least a portion of the r-pygas stream in an absorber-stripper system to remove at least a portion of the quantity of CO₂ from the r-pygas stream, thereby producing a CO₂-depleted r-pygas stream; and
(c) introducing at least a portion of the CO₂-depleted r-pygas stream into a separation process to thereby produce one or more recycled content chemical product(s) and co-product(s).

11. The process of claim 10, wherein the CO₂-depleted r-pygas stream comprises not more than 1000 ppm CO₂.

12. The process of claim 10, wherein the CO₂-depleted r-pygas stream is depleted in sulfur and/or sulfur-containing compounds.

13. The process of claim 10, wherein the waste plastic comprises not more than 10 percent by weight polyesters.

14. The process of claim 10, wherein the waste plastic comprises at least 80 percent by weight polyolefins.

15. The process of claim 10, wherein the recycled content chemical product(s) and co-product(s) comprise one or more olefins, alkanes, aromatics, hydrogen (H₂), paraffins, gasoline, and/or C5+ hydrocarbons.

16. The process of claim 10, wherein the recycled content chemical product(s) and co-product(s) comprise r-ethylene, r-propylene, r-butylene, r-benzene, r-toluene, r-xylenes, and/or r-styrene.

17. A process for recovering heat from a pyrolysis effluent stream, the process comprising:
(a) pyrolyzing waste plastic to thereby produce the pyrolysis effluent stream;
(b) cooling and at least partially condensing at least a portion of the pyrolysis effluent stream via indirect heat exchange with:
(i) a heat transfer medium (HTM), thereby warming the HTM; and/or
(ii) a stripper column reboiler;
(c) feeding at least a portion of the cooled and at least partially condensed pyrolysis effluent stream to a separator to thereby produce a pyrolysis gas (pygas) stream and a pyrolysis oil (pyoil) stream;
(d) treating at least a portion of the pygas stream in an absorber-stripper system to thereby produce a purified pygas stream; and
(e) optionally, using at least a portion of the warmed HTM to provide heating to one or more of:
(i) a rich solvent stream within the absorber-stripper system;
(ii) a liquefication process; and/or
(iii) a pyrolysis feedstock preheating process.

18. The process of claim 17, wherein the pyrolysis effluent stream has a temperature of 500° C. to 800° C. after the pyrolyzing (a).

19. The process of claim 17, wherein the heat transfer medium (HTM) comprises water/steam, oils, siloxanes, molten metal, molten salt, and/or combinations thereof.

20. The process of claim 19, wherein the HTM comprises an oil selected from the group consisting of synthetic oil, refined oil, or a combination thereof.
